SEQUENCE LISTING (2 sequences)

SEQ ID NO: 1 - DNA, 10919 bp, synthetic construct
  source 1..10919: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgcggccgcattctggtaccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgaggcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttcatgcatgatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccacaccagccaccaccttctgataggcagcctgcacctgaggacaattttaattaagcgatcgcctactgaaagtcgaaggacagcttccggggagtcatgaactctttcactatctcatccgtgacctccttgcgccgggcctggtgctctgcgatcaagggctgcagaacctctatgagtgccttcttgagctcaccggtgagcatggctccgctggtgtaatccttcctgatctgctcgagcttgtcgtcgtcctcgaggaagaaggtcaggtacatgaaagacacgtccacatcacagttgcccccaaactgcctgtgctcctcgatggtgtctctccctccagaaaacgcatgcttattgaccttggttttgatctgcttggccgtgtcggtgaggaagatggaggagttggggtcgctggcactcattttggtctgggcgccctgcagggctgggaagaaggtggagtgcagcagggctggtttaggatagccgatcctgggggcgacgtcccttgtcattctaaagtaaggatcctggtcaatggcacatgggataaggcactggatatccgtcctgtctcggaagatctgtgggaatgagttgctgaaggagggagcagcctggatggcaggaaaactgatcttcccaatgcagtcgctgtcagtgaagccgaaaatgcctttcacttggttgaaggtaacatgcttttgaatcttcaccacatttttgtagaaacctgagctcatccccatgtagtccaggtcagagaatatgaaagtcttgttgatgtcaaagccacaggcgatgatgtccttggcattctccacagcatagctataggcctggtccagggtcaggtccttccacagatacttctcgtcatccgtcatctggatgaccaagggcacgttaaatacatcctggagccactttgtgaaaataaatggaatgaggtgacctacatgcattgcttcagaagaggggccccggcccgtgtacagataaaatggcttcttattttcataggcatcaagaacctgattcatatctctgtgtgagaagaagatgcctctgcgcaggaagtggtgtggtctttggccggtggctctctctattcggtttattagctctttgtcaattttactacttccaaaccgaacaatgagcttatcgtagtctatgccttttgcactgcttgtctgtactgtccatgggtccacaaaatcctcttcagcttctgtggcatctgggccatgattactggtaggtgctgggttccctggaggacagtcagccttgtaatcctcccccgcggcagctttgtagctcatttttaatgacaccaacatctttactgcagaatcaatttcatcctttgacgcatttcccgctttgagggaccttacgagctccccttgtgtggcgatgctgttgaacagctccagcagagatgcgggctcactgttgggcatggtggcgatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggcgcgcctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttggggatatccacccctaggaggtatcgcgacggatggatccaagaaccagcccgggcggtggagctcgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggacagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - protein, 471 aa, Homo sapiens
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

(19) United States
(12) Patent Application Publication
(10) Pub. No.: US 2026/0150820 A1
(43) Pub. Date: Jun. 4, 2026

(54) HUMAN TRYPTOPHANYL-TRNA SYNTHETASE TRANSGENIC MOUSE MODELS

(71) Applicants: THE UNIVERSITY OF HONG KONG, Hong Kong (CN); Centre for Virology, Vaccinology and Therapeutics Limited, Hong Kong (CN)

(72) Inventors: Lee Lee TENG, Hong Kong (CN); Sze Ming Kenneth LI, Hong Kong (CN); Man Lung YEUNG, Hong Kong (CN); Lilong JIA, Hong Kong (CN)

(73) Assignees: THE UNIVERSITY OF HONG KONG, Hong Kong (CN); Centre for Virology, Vaccinology and Therapeutics Limited, Hong Kong (CN)

(21) Appl. No.: 19/404,690

(22) Filed: Dec. 1, 2025

Related U.S. Application Data
(60) Provisional application No. 63/727,241, filed on Dec. 3, 2024.

(51) Int. Cl. A01K 67/0278; C12N 9/00

(52) U.S. Cl. CPC A01K 67/0278; C12N 9/93; A01K 2217/072; A01K 2227/105; A01K 2267/0337; C12Y 601/01002

(57) ABSTRACT

The present disclosure relates to animal models that express human tryptophanyl-tRNA synthetase (hWARS) knock-in. These animal models are highly susceptible to enterovirus infection, including but not limited to EV-D68 and EV-A71 infection. Also disclosed are screening methods and compounds that utilize these animal models.

### RELATED APPLICATION

The present application claims priority to U.S. Provisional Application Ser. No. 63/727,241 filed Dec. 3, 2024 which is incorporated by reference in its entirety.

### SEQUENCE LISTING

The instant application contains a Sequence Listing which has been submitted electronically in XML file format and is hereby incorporated by reference in its entirety. Said XML copy, created on Nov. 26, 2025, is named 10030_012874-US1_SL.xml and is 13,973 bytes in size.

### 1. FIELD

The disclosure is generally directed to animal models that express human tryptophanyl-tRNA synthetase (hWARS). These animal models are highly susceptible to enterovirus infection. Screening methods and compounds that utilize these animal models are disclosed.

### 2. BACKGROUND

Enterovirus is a positive single-stranded RNA virus first isolated in California, United States in 1969 (1). This virus is one of the major causative agents for hand, foot, and mouth disease (HFMD). It is also one of the most common agents causing viral community-acquired pneumonia (2). Several non-polio enteroviruses, including enterovirus 71 (EV-A71) and enterovirus 68 (EV-D68), are capable of inducing severe neurological diseases including aseptic meningitis, acute flaccid paralysis, and fatal neurogenic pulmonary edema (3). EV-A71 is a common cause of HFMD in infants and children, and can cause life-threatening brainstem encephalitis, meningitis, and polio-like paralysis. EV-A71 is endemic to the Asia-Pacific region, including Hong Kong, which has seen an increase in the number of infections in the last two decades with yearly large-scale outbreaks. EV-D68, another emerging enterovirus, was first identified as a causative agent causing rare respiratory illness in the early 2000s. Attention was not given to the diseases caused by this enterovirus until 2014, when a severe respiratory disease outbreak in children occurred, which was linked to a rise in cases of acute flaccid myelitis (AFM) caused by the EV-D68. Since then, cases of EV-D68 respiratory disease and associated AFM have increased worldwide with outbreaks of EV-D68 following a biennial, seasonal pattern. In addition to EV-D68 and EV-A71, neurological diseases caused by other enteroviruses have also been reported sporadically. The increasing threat to human health posed by enteroviruses underscores the importance of countermeasures needed to target this broad group of viruses.

Disease pathogenesis is largely determined by viral tissue tropism, which is influenced by the availability of specific receptors on surface of host cell. In the case of enterovirus, the cell entry process is often complicated by involvement of multiple receptors and entry factors (4). Additionally, most enteroviruses capable of causing severe diseases do not share the same set of receptors and entry factors for infection, making it challenging to study disease pathogenesis. For example, sialic acid and intercellular adhesion molecule 5 (ICAM-5) have been postulated to be the main receptors for cell entry of EV-D68. However, studies have shown that many contemporary clinical EV-D68 strains could initiate infection even in the absence of sialic acid. The reliance on an alternative cellular receptor alone does not fully explain the viral tropism. This is because the expression of ICAM-5 is only expressed on the surface of telencephalic neurons of the central nervous system (CNS). The cell-type specific expression of ICAM-5 is unlikely to fully account for the broader neurotropism of EV-D68, as infections could be detected in ICAM-5 non-expressing neurons, such as distal axons of motor neurons. It is possible that another receptor is required for the EV-D68 to access the CNS. Finally, it is important to validate the requirement of sialic acid and ICAM-5 in EV-D68 infection using animal models.

EV-A71 is one of the most extensively studied non-polio enteroviruses. Nine cell entry factors, including human scavenger receptor class B member 2 (hSCARB2), human P-selectin glycoprotein ligand 1 (hPSGL1), annexin II (Anx2), heparan sulfate, sialic acid, dendritic cell-specific ICAM3-grabbing non-integrin, vimentin, nucleolin etc., have been reported so far (5). Among them, the hSCARB2, a ubiquitously expressed type III transmembrane scavenger receptor protein localized on the lysosomal membrane, is believed to be the main receptor for cell entry of EV-A71. In vitro evidence proposed that the hSCARB2 could mediate binding, internalization, and uncoating of EV-A71. However, a recent study suggested that the absence of surface expression of hSCARB2 in most of EV-A71-susceptible cells highlighting the possibility that another receptor is responsible for enterovirus attachment to the cell and probably for internalization of virions (6).

Small laboratory animals, including mouse, are naturally non-permissive to enteroviral infections (7). To overcome this, various strategies, including the use of mouse-adapted viruses or immunocompromised mice models, have been adopted in an attempt to make them susceptible to enteroviruses (7). However, these approaches have limitations. Accumulated mutations during mouse adaptation for the generation of mouse-adapted viruses and the lack of specific types of immune cells in immunocompromised mouse models limited their applications in studying virus biogenesis and host immune responses, respectively. Moreover, murine homologs of human cellular receptors have been shown to be incompatible with the binding of surface proteins of enteroviruses. Therefore, careful validation of the data obtained from these models will be required. A more relevant approach to studying enteroviral infection in vivo may involve transgenic models with overexpression of known human receptors. Several independent laboratories have generated transgenic mice expressing the hSCARB2 gene. However, these hSCARB2-transgenic mouse models have shown limited success in supporting EV-A71 infection. While the hSCARB2-transgenic mice remained resistant to EV-A71 in adulthood, neonatal hSCARB2-transgenic mice developed symptoms resembling HFMD and paralysis after being challenged with EV-A71. Tissue tropism characterization of the EV-A71-infected hSCARB2-transgenic mice revealed that infection mainly occurred in skeletal muscles. Although EV-A71 antigens could also be detected to a lesser extent in other organs, including the brainstem, spinal cord, and intestine, it remains unclear whether paralysis is a result of excessive damage to the infected skeletal muscles or neuronal damage in the infected brainstem, and/or spinal cord. Another well-characterized EV-A71 receptor is hPSGL1, primarily expressed on leukocytes. Its role in microinvasion has been proposed due to the detection of hPSGL1 expression in both neurons and glial cells. However, transgenic mice expressing hPSGL1 have failed to support infection of clinical strains of EV-A71. Therefore, development of a more robust animal model is essential for studying enteroviral infections.

### 3. SUMMARY

Enterovirus (EV) is an emerging infectious virus causing over 1 billion of enteroviral infections annually worldwide. Of particular concern, several EVs, including EV-A71, are capable of inducing severe diseases. Attempts have been made to develop anti-viral drugs and vaccines for treating enteroviral infections. However, the key research challenge in the field is a lack of an appropriate animal model for enterovirus infection which makes evaluation of in vivo effects of these treatment options impossible. Provided herein is a novel entry factor, hWARS, which expression supports multi-species enteroviral infections. A transgenic mouse model expressing the hWARS is generated using CRISPR technology. Challenge of the neonatal hWARS-transgenic mice with EV-A71 showed symptoms that resemble human pathologies. Notably, adult hWARS-transgenic mice exhibit high susceptibility to EV-A71 infection. The disclosed animal model is useful for studying the enteroviral infection. In one embodiment, the susceptibility of the hWARS-transgenic mouse model is used to test infection from different species of enteroviruses. In certain embodiments, anti-enteroviral drugs are tested in the hWARS-transgenic mouse model. Lastly, the susceptibility of enteroviral infection of the disclosed hWARS-transgenic mouse model at their adulthood provides a unique platform for vaccine development. In one embodiment, the hWARS-transgenic mice are immunized and the efficacy of the EV-A71 vaccine is tested.

Provided herein is a transgenic mouse comprising a hWARS vector that expresses at least a portion of a hWARS gene and functional/structural variants thereof, in one or more tissues and exhibits an increased susceptibility to enterovirus infection as compared to a mouse without the hWARS gene.

In one embodiment, the hWARS gene is a wild-type gene.

In one embodiment, the hWARS vector is targeted to a mouse ROSA26 gene.

In one embodiment, the hWARS vector is shown in FIG. 3.

In one embodiment, the hWARS vector comprises an amino acid sequence of SEQ ID NO:2.

In one embodiment, the transgenic mouse is a mouse model for enterovirus infection.

In one embodiment, the enterovirus infection is EV-D68, EV-A71 or a combination thereof.

In one embodiment, the enterovirus infection is a disease or condition selected from the group consisting of severe neurological disease, aseptic meningitis, acute flaccid paralysis, fatal neurogenic pulmonary edema, hand foot and mouth disease (HFMD), brainstem encephalitis, meningitis, polio-like paralysis, severe respiratory disease, and acute flaccid myelitis (AFM).

In one embodiment, the hWARS gene and functional/structural variants thereof is expressed at least in one tissue comprising brain, muscle, and lung.

In one embodiment, the transgenic mouse comprises a hWARS knock-in mutation.

In certain embodiments, the present disclosure provides cells, cell lines, or tissues derived from or isolated from the transgenic animals of the present disclosure. The cells or cell lines may be useful in studies of enteroviral infection or in screening or assessing the effect of potential compounds, drugs or agents.

In one embodiment, the mouse is treated with a compound which lowers the level of susceptibility to enterovirus infection.

In one embodiment, the enterovirus infection is treated by administering small molecules, antibiotics, antiviral agents or antibodies.

Provided is a vector comprising a CMV promoter-Kozak-human WARS CDS-rBG pA cassette.

Provided is a method of screening for compounds having an effect on treating enterovirus infection comprising: (i) administering the compound to a transgenic mouse that expresses a portion of a hWARS gene and functional/structural variants thereof, in one or more tissues with an increased susceptibility of enterovirus infection as compared to that in a wild-type mouse, wherein the mouse comprises a hWARS knock-in vector; and (ii) determining the effect of the compound on treating enterovirus infection in the transgenic mouse, relative to a control mouse not treated with the compound or a control mouse without the hWARS knock-in vector treated with the compound; and (iii) identifying the compound.

Provided is a method of treating a disease or condition in a subject in need thereof, said method comprises administering an effective amount of a compound identified in the disclosed method, said disease or condition is an enterovirus infection which is selected from the group consisting of severe neurological disease, aseptic meningitis, acute flaccid paralysis, fatal neurogenic pulmonary edema, hand foot and mouth disease (HFMD), brainstem encephalitis, meningitis, polio-like paralysis, severe respiratory disease, and acute flaccid myelitis (AFM).

### 4. BRIEF DESCRIPTION OF THE FIGURES

The patent or application file contains at least one drawing executed in color.

FIGS. 1A-B. Inoculation of 10⁶ EV-A71 [infectious dose/weight of mouse (gram)] into hWARS-transgenic mouse at 10 days of age showed a marked reduction in body weight increment (FIG. 1A: light grey) and the onset of symptoms, including bilateral rear limb paralysis with marked difficulty in walking, compatible with neurological disorders at day 6 post-infection (FIG. 1B: dark grey) and displayed symptom-free phenotypes (FIG. 1B, right).

FIGS. 2A-B. (A) Immunohistopathological and qPCR analyses confirmed the presence of infection in various organs, including brain, muscle, and lung. (B) Plaque assay for EV-D68-infected hWARS-tg mouse Brain/Lung/Muscle organs.

FIGS. 3A-B. (A) Targeting vector of the hWARS-transgenic mouse. (B) Targeting vector of the hWARS-transgenic mouse with nucleic acid positions corresponding to various regions.

FIG. 4. Nucleic acid sequence of the hWARS vector (SEQ ID NO:1). The vector rBG PA region is from 2788 bp to 3331 bp, the vector Kozak-human WARS CDS region is from 3332 bp to 4758 bp, and the vector CMV promoter region is from 4759 bp to 5342 bp. The nucleic acid positions corresponding to various regions are indicated in FIG. 3B.

FIG. 5. Amino acid sequence of hWARS (SEQ ID NO:2).

### 4.1. DEFINITIONS

The term “variant” or “variants” in reference to a protein comprises one or more mutations in the parent protein, such as a substitution, a deletion, an insertion and/or an addition to the parent protein. The “variant” or “variants” within this definition in reference to a protein still have the hWARS activity. For example, a variant protein may have one or more mutations at the wild-type hWARS protein (SEQ ID NO:2) that does not affect the activities of hWARS. For instance, a substitution at a conservative amino acid residue with another one having similar chemical properties, is unlikely to affect the activity of said protein. Two amino acid sequences are “substantially homologous” when about 70-80%, 80%-90%, 90%-95%, 95%-99% of the amino acid residues are identical, or represent conservative substitutions.

Modified protein expressed from the sequence of NCBI reference sequence: NM_004184.4 can also be employed in this disclosure. Useful modifications within this context include, but are not limited to, those that alter post-translational modifications, size or active site, or that fuse this protein or portions thereof to another protein. In one embodiment, a structural/functional variant is at least 75% identical to SEQ ID NO:2. In one embodiment, a structural/functional variant is 80% identical to SEQ ID NO:2. In another embodiment, a structural/functional variant is 90% identical to SEQ ID NO:2. In one embodiment, a structural/functional variant is 95% identical to SEQ ID NO:2. Modified protein expressed from the sequence of NCBI reference sequence: NM_004184.4 can be employed in this disclosure.

The term “variant” or “variants” in reference to a nucleic acid may be made by introducing one or more mutations in the nucleic acid having the nucleic acid sequence of NM_004184.4. In one embodiment, a structural/functional variant is at least 75% identical with the nucleic acid sequence of NCBI reference sequence: NM_004184.4. In one embodiment, a structural/functional variant is 80% identical with the sequence of NCBI reference sequence: NM_004184.4. In another embodiment, a structural/functional variant is 90% identical with the sequence of NCBI reference sequence: NM_004184.4. In one further embodiment, a structural/functional variant is 95% identical with the sequence of NCBI reference sequence: NM_004184.4 In one embodiment, the nucleic acid variant expresses a protein variant that retains hWARS activities. Two nucleic acid sequences are “substantially homologous” when about 75%-80%, 80%-90% or 90%-95%, about 95%-99% of the nucleotides match over the defined length of the nucleic acid sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, The terms “enterovirus” and “enteroviruses” refer to RNA viruses which are of the piconarviridae family as previously described [Fields Virology (1996), 3rd Edition, Publ: Lippincott S. Raben, Chapter 22]. Enteroviruses have sense RNA and non-enveloped virus particles. Enteroviruses include, without limitation, polioviruses, Coxsackie A viruses, Coxsackie B viruses, echoviruses, and enterovirus types 68, 69, 70 and 71. Polioviruses are exemplified, but not limited to poliovirus types 1, 2, and 3. Coxsackie A viruses include, without limitation, Coxsackie virus types A1, A2, A3, A4, A5, A6, A7, A8, A9, A10, A11, A12, A13, A14, A15, A16, A17, A18, A19, A20, A21, and A24. Coxsackie B viruses are exemplified by Coxsackie virus types B1, B2, B3, B4, B5, and B6. Echoviruses include, by way of example, echovirus types 1, 2, 3, 4, 5, 6, 7, 8, 9, 11, 12, 13, 14, 15, 16, 17, 18, 19, 20, 21, 22, 23, 24, 25, 26, 27, 29, 30, 31, 32, 33, and 34.

The term “animal” used herein refers to any mammalian animal except a human. It also includes an individual animal in all stages of development, including embryonic and fetal stages. A “transgenic” animal is any animal containing cells that bear genetic information received, directly or indirectly, by deliberate genetic manipulation at the subcellular level, such as by the infection or genetic microinjection with recombinant virus. The transgenic animals of the present disclosure are other than human. Farm animals (pigs, goats, sheep, cows, horses, rabbits and the like), rodents (such as rats and mice), and domestic pets (for example, cats and dogs) are included in the scope of this disclosure. In one embodiment of the present disclosure, the transgenic mammal as used may be any one selected from the group consisting of mouse, rat, pig, goat, sheep, cow, horse, rabbit, cat and dog. In one embodiment, the transgenic mammal as used is any one selected from the group consisting of mouse, rat, pig, goat, sheep, cow, horse and rabbit.

The term “preventing” or “prevention” refers to a reduction in risk of acquiring or developing a disease or disorder (i.e., causing at least one of the symptoms of the disease not to develop) in a subject that may be exposed to a disease-causing agent, or predisposed to the disease in advance of disease onset.

“Therapeutically effective amount” means that amount of a drug, compound, or pharmaceutical agent that will elicit the biological or medical response of a subject that is being sought by a medical doctor or other clinician. The phrase “therapeutically effective amount” is used herein to mean an amount sufficient to reduce by at least about 30 percent, more preferably by at least 50 percent, most preferably by at least 90 percent, a clinically significant reduction in a symptom or symptoms associated with a disease or disorder.

The term “treating” or “treatment” of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or infection, either physically, (e.g., stabilization of a discernible symptom), physiologically, (e.g., stabilization of a physical parameter), or both. In a further embodiment, “treating” or “treatment” relates to slowing the progression of a disease.

### 5. DETAILED DESCRIPTION

Enterovirus (EV) is a non-enveloped, positive-sense single stranded RNA virus. It belongs to the genus Enterovirus under the family Picornaviridae. There are 71 human enterovirus serotypes causing a wide range of illness from cold-like respiratory diseases to severe central nervous system diseases. Of which, enterovirus A71 (EV-A71) is the major causative agent of hand, foot, and mouth disease (HFMD) globally. The infection can be complicated by meningitis and acute flaccid paralysis. Another serotype, enterovirus D68 (EV-D68), infects infants and children usually, has caused epidemics of severe respiratory illness and acute flaccid myelitis (AFM) associated with mortality in the United States. The pathogenesis of EV-A71 and EV-D68 is not fully understood so far and there is currently no vaccine available for the two viruses.

Although there are numerous animal models for enterovirus infection, differential pathogenicity was displayed, which cannot truly mimic the viral infections in human. In particular, neurological symptoms such as limb paralysis were only demonstrated in neonatal mice. Besides, some other mouse models require the need of mouse-adapted enterovirus strains for infection. This hinders the enterovirus therapeutics development due to the lack of a suitable animal model for studying the disease pathogenesis as well as for evaluating antiviral efficacy. Human tryptophanyl-tRNA synthetase (hWARS) is an interferon gamma (IFN-γ)-inducible cellular entry factor for EV-A71 and EV-D68.

Provided herein is a human WARS knock-in (KI) transgenic mouse model using CRISPR/Cas 9-mediated genome engineering. This KI mouse model was shown to be highly susceptible to enterovirus infection in both neonatal and adult mice by different infection routes. Moreover, neurological symptoms as shown in human clinical manifestation of EV-D68 and EV-A71 infections such as limb paralysis can also be demonstrated in this animal model, with viral RNAs and live viruses detected in various tissues. Also, mouse-adapted viral strains are not needed for this mouse model. These factors make this mouse model ideal for studying EV-A71 and EV-D68 infections.

The present disclosure describes a hWARS knock-in transgenic mouse model which is highly susceptible to EV-D68 and EV-A71 infection, with symptoms resembling human infections. Current animal models can only display paralytic phenotype in neonatal mice upon infection while hWARS KI mice model exhibits neurological symptoms and lethality in both neonatal and adult mice. This greatly improves the practical use of the mouse model in studying disease pathogenesis and evaluating vaccine and antiviral efficacy. The hWARS knock-in transgenic mouse model is a new concept. The hWARS KI mouse model allows the recapitulation of pathologies developed in patients with severe enterovirus infections, which is a more suitable animal model for enterovirus in vivo studies.

Animal model are useful, for example: (i) Generation of knock-in animal models for screening; (ii) method of inducing disease states; (iii) studying the disease state; and (iv) assaying for compounds. These animal models can be used to study mechanisms and progression of aforementioned disease state, as a function of diet, treatment with drugs to be screened for efficacy or undesirable side effects, and social environmental effects.

The disclosure also provides a nucleic acid molecule, encoding the protein hWARS or variants thereof, and fragments thereof. In one embodiment, the protein hWARS is encoded by the nucleotide sequence NCBI reference sequence: NM_004184.4. Anyone skilled in the art can prepare the protein hWARS having amino acid sequence of SEQ ID NO:2 according to the standard method or technologies.

Another object of the present disclosure is to provide a construct for expression of the protein hWARS in various tissues of a non-human mammal comprising: (a) a specific promoter; (b) a transgene having the nucleotide sequence coding for the protein hWARS; and (c) a polyadenylation signal sequence for stabilizing the expression of the transgene in tissues; wherein (a), (b) and (c) are operably linked in order from 5′-terminal to 3′ terminal. In one embodiment, the transgene for expression of the protein hWARS is NCBI reference sequence: NM_004184.4. In one embodiment, the protein hWARS comprises the amino acid sequence of SEQ ID NO:2.

In one aspect, the present disclosure relates to a recombinant nucleic acid molecule comprising a nucleic acid sequence encoding hWARS under the control of a regulatory sequence directing its expression. Transcriptional and translational control sequences are regulatory sequences, such as promoters, enhancers, polyadenylation signals, terminators, and the like, that provide for the expression of a coding sequence in a host cell.

In one embodiment, the nucleic acid sequence in the recombinant nucleic acid molecule present in the non-human animal is operably linked to a regulatory sequence directing its expression. “Operably linked” when describing the relationship between two polynucleotide sequences, means that they are functionally linked to each other. For example, a promoter is operably linked to a coding sequence if it controls the transcription of the sequence. As a regulatory sequence commonly used promoter elements as well as enhancers may be used. Generally, such expression regulation sequences are derived from genes that are expressed primarily in the tissue or cell type chosen, although secondary expression in other tissue and/or cell types is acceptable if expression of the recombinant DNA in the transgene in such tissue or cell type is not detrimental to the transgenic animal.

The recombinant nucleic acid molecules will usually also comprise downstream expression regulation sequences to supplement tissue or cell-type specific expression. The downstream expression regulation sequences include polyadenylation sequences (either from the endogenous gene or from other sources) and sequences that may affect RNA stability as well as enhancer and/or other sequences which enhance expression.

In certain embodiments, the present disclosure provides a cassette for expression of hWARS. The cassette comprises: (i) a promoter; (ii) a transgene having nucleic acid sequence coding for hWARS; and (iii) a polyadenylation signal sequence for stable expression of transgene; and wherein (i), (ii) and (iii) are operatively linked in order from 5′-terminal to 3′-terminal.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. In certain embodiments, if a gene that one desires to insert into a recombinant DNA molecule does not contain an appropriate start signal, such a start signal can be inserted in front of the gene.

In one embodiment, the present disclosure provides a targeting vector comprising a CMV promoter-Kozak-human WARS CDS-rBG pA cassette. The cassette contains the human WARS gene.

In certain embodiment, the promoter used may be anyone that drives expression of hWARS. In one embodiment, the promoter is CMV promoter. Generally, any promoter known to direct the expression of a protein in the art, such as EF-1α, CAG, PGK, UBC may be used.

In certain embodiments, any polyadenylation signal sequence for stabilizing the expression of the transgene in the transgenic animal may be used. In one embodiment, the sequence for stabilization is bovine GH polyadenylation signal sequence (bGH poly(A)).

In certain embodiments, hWARS transgene was constructed for used in microinjection to produce a transgenic animal.

Several methods are known in the art to introduce a recombinant nucleic acid molecule into an embryo of a non-human animal. These include, for example, microinjection into a nucleus of a fertilized ovum, retroviral transfection of embryonal cells, and transfection of embryonic stem cells.

In one embodiment, the present disclosure provides a non-human transgenic animal that produces hWARS wherein the transgenic mammal has stably integrated into its genome the exogenous gene cassette.

In one embodiment, the present disclosure provides a non-human transgenic animal that produces hWARS having NCBI reference sequence: NM_004184.4, or a variant thereof.

In certain embodiments, the disclosure provides a non-human transgenic animal that is highly susceptible to enterovirus infection.

In one embodiment, provided is an animal model for a disease or disorder related to enteroviral infection. In particular, the present disclosure relates to a transgenic non-human animal comprising a recombinant nucleic acid molecule containing a nucleic acid sequence encoding hWARS, wherein said animal displays one or more symptoms of a disease or disorder related to enteroviral infection. In certain embodiments, the enterovirus is EV-A71 and EV-D68.

The present disclosure provides systems and methods in which the finding that tryptophanyl-tRNA synthetase (WARS) is a cellular surface receptor for enteroviruses, including enterovirus EV-A71 and EV-D68. In certain embodiments, the present systems and methods are utilized in the treatment of EV-71 and EV-D68 infections and in the generation of cell culture and animal models for human EV-A71 and EV-D68 infections.

In certain embodiments, provided are compositions and methods that reduce the expression of WARS and/or translocation of WARS to the cell surface or block WARS at the cell surface. Such compositions and methods can be useful in the treatment and prevention of infection by EV-A7, EV-D68 and other enteroviruses.

In certain embodiments, provided herein are induction of the expression of WARS in animals and cultured cells can render such animals and cells susceptible to EV-A71 and EV-D68 infections, thereby providing non-human models of human disease. Such animal models and cultured cell models can be utilized in the development of treatment and/or preventative modalities, in addition to aiding in the development of diagnostic tools.

In certain embodiments, the disease or disorder is severe neurological disease, aseptic meningitis, acute flaccid paralysis, fatal neurogenic pulmonary edema, hand foot and mouth disease (HFMD), brainstem encephalitis, meningitis, polio-like paralysis, severe respiratory disease, and acute flaccid myelitis (AFM).

The present disclosure relates to the use of the transgenic animals for the design and screening of drugs.

In one embodiment, the screening of drug candidates for the prevention or treatment of a disease or disorder comprising the administration of one or more drug candidates directly to the transgenic animal; and evaluating the effect of the drug candidate(s) on the animal.

By “drug candidate,” “candidate compound,” “test compound,” “agent,” or “therapeutic agent,” as used herein, means any molecule, e.g. a protein or pharmaceutical, i.e., a drug, with the capability of treating or preventing one or more symptoms of enteroviral infection, e.g., a transgenic animal having a sequence that encodes hWARS, which has been contacted with said drug candidate, candidate compound, test compound, agent, or therapeutic agent, relative to a transgenic animal that has not been contacted with the drug candidate, candidate compound, test compound, agent, or therapeutic agent.

Useful compounds/agent for testing includes but are not limited to Pleconaril (picornaviruses viral capsid inhibitor), Rupintrivir (viral protease inhibitor), Remdesivir (viral RNA replication inhibitor), Vemurafenib (viral RNA replication inhibitor) and Pocapavir (picornaviruses viral capsid inhibitor).

Those skilled in the art will recognize that numerous modes of administration. Modes of administration include, but are not limited to, topical application, intra- and subdermal injection, aerosol administration, and transdermal administration (e.g., in a carrier, such as DMSO). The selection of a particular mode of administration will depend on the composition. Similarly where a potential therapeutic is expected to be administered topically as opposed to systemically, the potential therapeutic will be screened using a topical application.

In certain embodiments, the screening methods are performed with the drug candidates provided as a collection of compounds. In one embodiment, the number and/or diversity of compounds within said collection is successively reduced in repeated screening rounds. Such collections are also commercially available, for example, from Pharmacopeia, Inc. or Chemical Diversity Labs, Inc.

In certain embodiments, the drug candidate(s) are small molecule(s). Methods for the synthesis, optimization, and testing of small molecules are well known in the art. In a further embodiment, the drug candidate(s) are antibodies, antibody conjugate(s). Antibodies play an increasing role in the treatment of disorders due to their high specificity. Once a target molecule with a critical role in the development of the particular disorder is identified, it is possible to generate either polyclonal or monoclonal antibodies capable of binding to it. The antibody can inhibit the detrimental function of the molecule either directly, for instance, by binding to the functional core, or by assigning the molecule to be degraded.

In certain embodiments, the drug candidates belong to different classes of drugs, such as but not limited to small molecules, antibodies or conjugated antibodies.

In one embodiment, the present disclosure provides method of screening for drugs, vaccines or other vehicles developed for the prevention and treatment of enterovirus infection.

### 6. EXAMPLES

### 6.1 Materials and Methods

6.1.1 Generation of hWARS Knock-In Mice

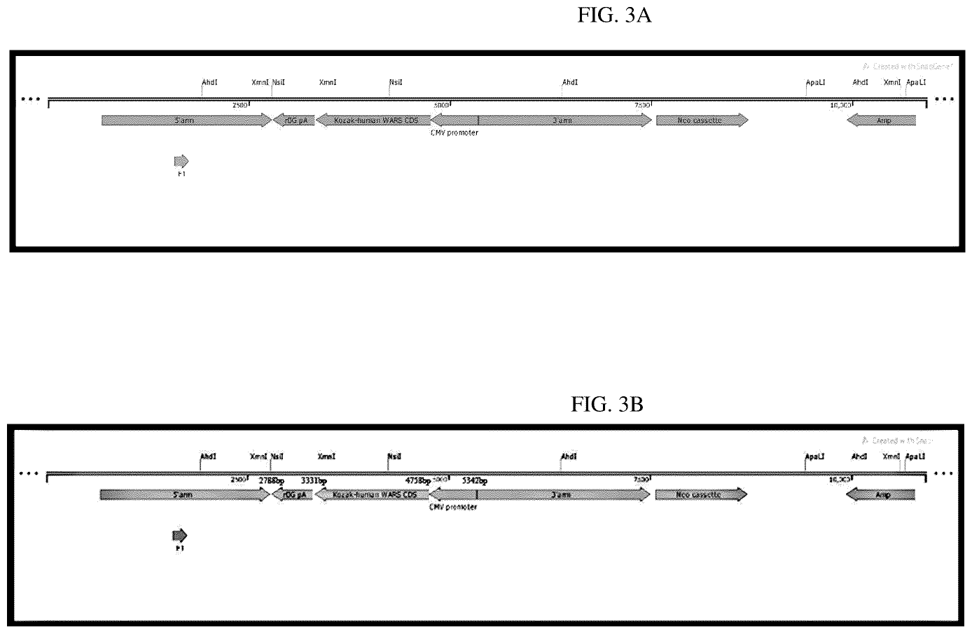

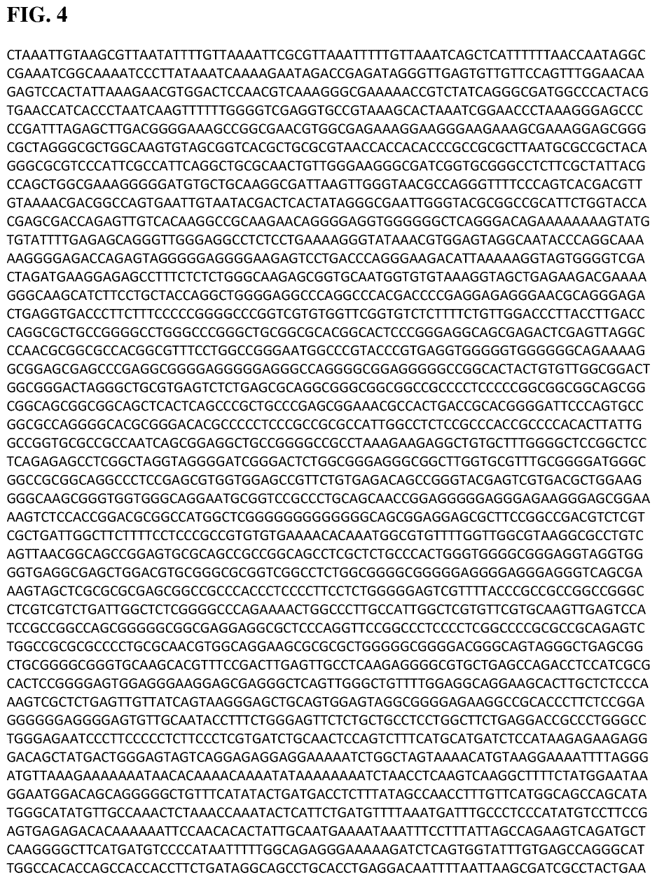

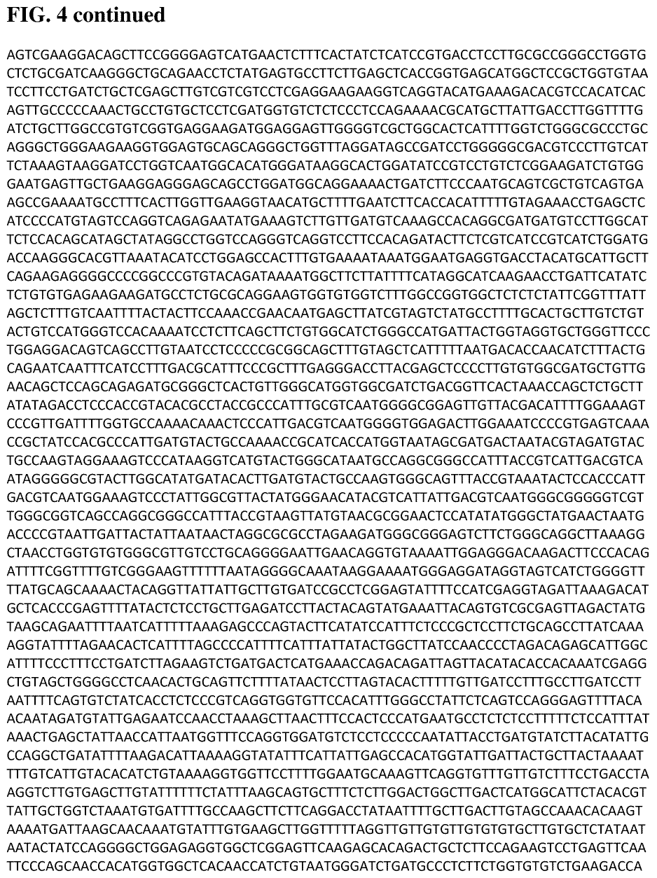

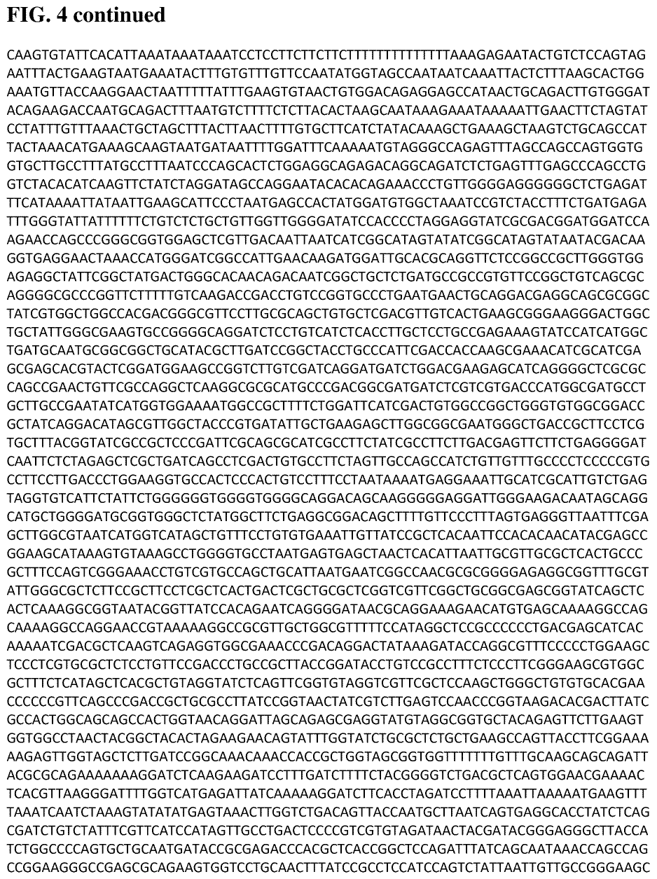

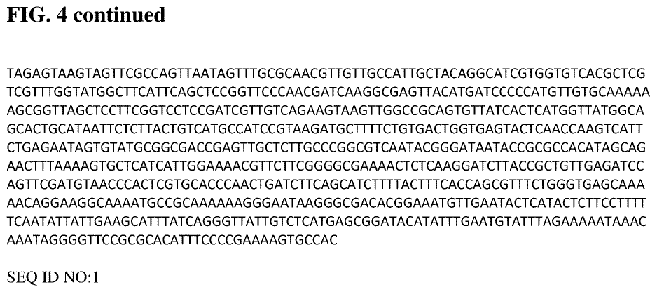

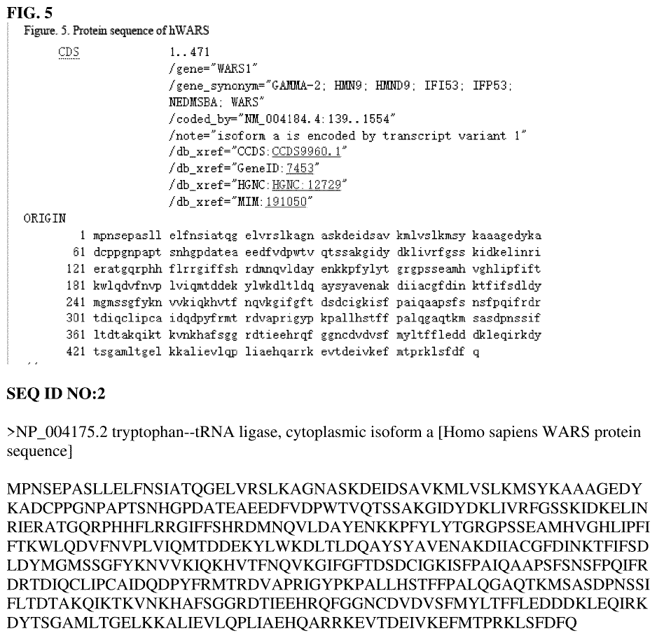

To generate hWARS KI mice, mouse genomic fragments containing homology arms (HAs) were amplified from bacterial artificial chromosome (BAC) clone by using high fidelity Taq DNA polymerase, and sequentially assembled into a targeting vector together with the “CMV promoter-Kozak-human WARS CDS-rBG pA” cassette containing the human WARS gene (NCBI reference sequence: NM_004184.4) from human chromosome 14. (FIG. 3 for targeting vector). For KI mice production, Cas9 and gRNA were co-injected into fertilized eggs with the targeting vector, in which the hWARS cassette is targeted to clone into intron 1 of mouse ROSA26 gene (NCBI reference sequence: NR_027008.1) on mouse chromosome 6 in reverse orientation. The pups were then genotyped by PCR followed by sequencing analysis to confirm the transgene.

### 6.1.2 Results and Discussion

The cell entry role of the hWARS during enteroviral infection has been first depicted in our research findings (7). While hWARS now has been recognized as one of the cellular receptors of EV-A71, it possesses some unique features that distinguish it from other receptors. For example, the cell surface expression of hWARS could be induced by interferon gamma (IFN-γ), a proinflammatory cytokine which production has been correlated with the disease severity of EV-A71 infection. Indeed, the hWARS represents the first IFN-inducible entry factor of enterovirus, which could be important in disseminating infections in cells and tissues that are typically not susceptible to infections. It should be noted that the hWARS-mediated EV-A71 infection could be independent of the expression of hSCARB2. This has been demonstrated by infection of EV-A71 in hSCARB2-knockout cells. In the absence of hSCARB2, these cells expressing hWARS remained susceptible to EV-A71 suggesting that the EV-A71 could enter cells via a hWARS-dependent, but hSCARB2-independent, entry route. The usage of hWARS for cell entry appears to be broadly used by many neurotropic enteroviruses, including the EV-A71 and EV-D68. Therefore, generating an animal model expressing the hWARS could help in understanding the disease-causing mechanism induced by these neurotropic enteroviruses.

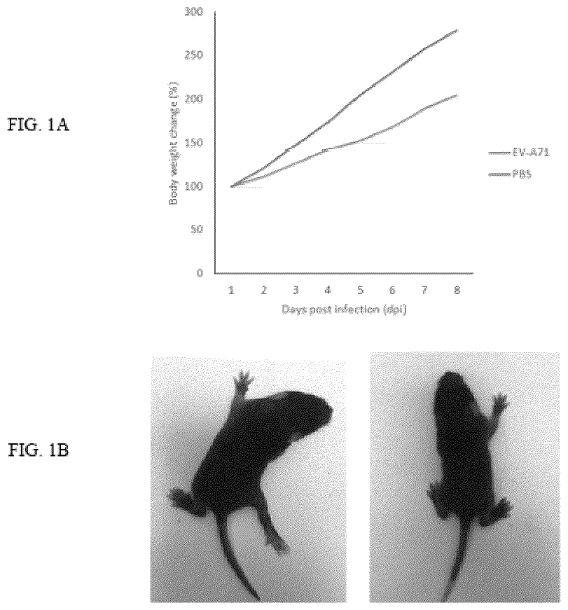

To investigate the in vivo cell entry effects of hWARS, hWARS was temporarily expressed in mice by using a lentiviral vector delivery system followed by challenging them with EV-A71. The mice expressing the hWARS developed symptoms resembled severe human infection, recapitulating EV-A71 neuropathogenesis. Viral RNAs and proteins could also be detected in the brain and muscle of EV-A71-infected mouse tissues suggesting that the hWARS-expressing mouse model could be useful in investigating enterovirus-induced neuropathogenesis. The in vivo results helped to establish a transgenic mouse model expressing the hWARS gene. This was done by specifically integrating the cDNA of hWARS into the Rosa26 locus using CRISPR/Cas9 technology. The correct integration site of the hWARS cDNA and its protein expression were validated by genomic DNA PCR and Western blot (WB) analyses of tissues extracted from the hWARS-transgenic mouse tails, respectively. Using this newly generated hWARS-transgenic mouse model, pilot experiments were performed using published infection conditions (7). Inoculation of 10⁶ EV-A71 [infectious dose/weight of mouse (gram)] into hWARS-transgenic mice at 10 days of age showed a marked reduction in body weight increment (FIG. 1A; light grey line) and the onset of symptoms, including bilateral rear limb paralysis with marked difficulty in walking, compatible with neurological disorders at day 6 post-infection (FIG. 1B; left). Comparatively, PBS-inoculated mice showed a steady increase in body weight (FIG. 1A; dark grey lines) and displayed symptom-free phenotypes (FIG. 1B; right). The disease phenotypes of the EV-A71-inoculated hWARS-transgenic mice match with that presented in other published mouse infection models (7, 8).

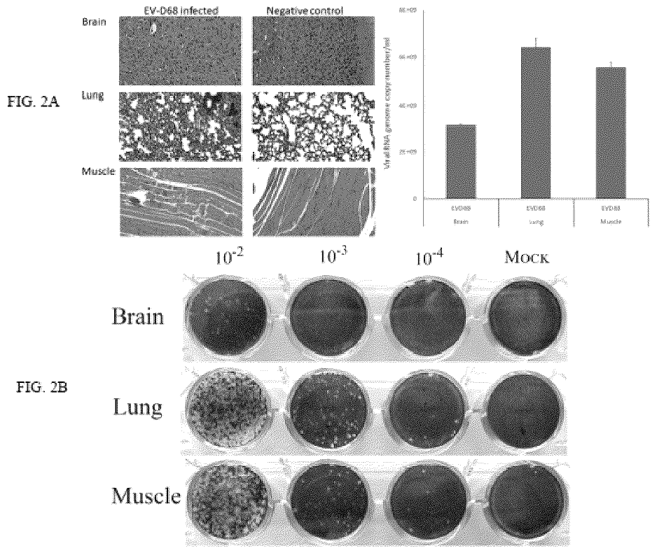

Our previous in vitro data demonstrated that the hWARS supports cell entry of a broad spectrum of enteroviruses (8). The disclosed hWARS-transgenic mouse model is useful in studying pathogenesis of another neurotropic enterovirus, EV-D68. Using the same infection conditions mentioned above, neonatal hWARS-transgenic mice (10-day-old) were inoculated with EV-D68. Surprisingly, an early and severe onset of disease in the hWARS-transgenic mice infected with EV-D68 were observed. Significant limb paralysis and shortness of breath were observed as early as 1 day post-infection. Within 5 days post-infection, all 3 EV-D68-infected mice had succumbed to the disease. The severe symptoms displayed in the infected mice correlated with the neuropathogenicity of the infecting enteroviruses (EV-D68>EV-A71 in clinical strains). Immunohistopathological and qPCR analyses confirmed the presence of infection in various organs, including brain, muscle, and lung (FIG. 2A). More importantly, infectious viruses were recovered from the EV-D68-infected brains confirming the permissiveness of enteroviruses in our mouse model (FIG. 2B). It should be noted that the original non-transgenic C57BL/6 mouse strain is non-susceptible to EV-D68, as confirmed by research and other studies (9, 10). Overall, the presently disclosed data shows that this novel hWARS-transgenic animal model supports neuronal infection of enteroviruses.

In one embodiment, assays or experimental procedures for evaluating the effectiveness of a screened compound includes the following:
1. The screening compound is dissolved in appropriate solvent that compatible for animal work.
2. The hWARS-transgenic mice is administered with a serial dose of compound through oral route or intraperitoneal route before/after EV-A71 or EV-D68 infection challenge at 10⁶ viral titer.
3. The effectiveness of the potential compound is evaluated by qPCR assay to determine viral copy number, WB assay to test viral protein amount or IFA assay to verify viral protein presence or absence in various mice organs.
4. The live virus presents inside different organs with/without compound treatment is determined by plague assay.

In one embodiment, the disclosure provides cells, cell lines and methods of using these cells or cell lines. The establishment of a primary cell line from a transgenic mouse offers a valuable in vitro model for detailed cellular and molecular investigations. This approach provides a controlled environment that minimizes variability inherent to in vivo systems, thereby enabling the generation of highly reproducible and consistent experimental results. Moreover, the use of primary cells facilitates the development of high-throughput screening platforms, which are challenging to implement in live animal models due to logistical and ethical constraints. Such in vitro systems are instrumental for rapid, large-scale testing of pharmacological agents, genetic modifications, or environmental conditions, ultimately accelerating the pace of research and reducing reliance on animal experimentation.

### Exemplary Products, Systems and Methods are Set Out in the Following Items:

1. A transgenic mouse comprising a hWARS vector that expresses at least a portion of a hWARS gene and functional/structural variants thereof, in one or more tissues and exhibits an increased susceptibility to enterovirus infection as compared to a mouse without the hWARS gene.
2. The mouse of item 1 wherein the hWARS gene is a wild-type gene.
3. The mouse of any one of the preceding items wherein the hWARS vector is targeted to a mouse ROSA26 gene.
4. The mouse of any one of the preceding items wherein the hWARS vector comprises a CMV promoter-Kozak-human WARS CDS-rBG pA cassette
5. The mouse of any one of the preceding items wherein the hWARS vector comprises a nucleic acid sequence of SEQ ID NO:1.
6. The transgenic mouse of any one of the preceding items which is a mouse model for enterovirus infection.
7. The transgenic mouse of any one of the preceding items wherein the enterovirus infection is EV-D68, EV-A71 or a combination thereof.
8. The transgenic mouse of any one of the preceding items wherein the enterovirus infection is related to a disease or disorder selected from the group consisting of severe neurological disease, aseptic meningitis, acute flaccid paralysis, fatal neurogenic pulmonary edema, hand, foot, and mouth disease (HFMD), brainstem encephalitis, meningitis, polio-like paralysis, severe respiratory disease, and acute flaccid myelitis (AFM).
9. The transgenic mouse of any one of the preceding items wherein the hWARS gene and functional/structural variants thereof is expressed at least in one tissue comprising brain, muscle, and lung.
10. The transgenic mouse of any one of the preceding items comprising a hWARS knock-in mutation.
11. The transgenic mouse of any one of the preceding items, wherein the mouse is treated with a compound which lowers the level of susceptibility to enterovirus infection.
12. The transgenic mouse of any one of the preceding items, wherein the enterovirus infection is treated by administering small molecules, antibiotics, antiviral agents, or antibodies.
13. A vector comprising a CMV promoter-Kozak-human WARS CDS-rBG pA cassette.
14. A method of screening for compounds that treat enterovirus infection comprising: (i) administering the compound to a transgenic mouse that expresses a portion of a hWARS gene and functional/structural variants thereof, in one or more tissues with an increased susceptibility of enterovirus infection as compared to that in a wild-type mouse, wherein the mouse that comprises a hWARS knock-in vector; and (ii) determining the effect of the compound on treating enterovirus infection in the transgenic mouse, relative to a control mouse not treated with the compound or a control mouse without the hWARS knock-in vector treated with the compound; and (iii) identifying the compound.
15. A method of treating a disease or disorder in a subject in need thereof, said method comprises administering an effective amount of a compound identified in the method of the preceding item, said disease or disorder is related to an enterovirus infection, the disease or disorder is selected from the group consisting of severe neurological disease, aseptic meningitis, acute flaccid paralysis, fatal neurogenic pulmonary edema, hand foot and mouth disease (HFMD), brainstem encephalitis, meningitis, polio-like paralysis, severe respiratory disease, and acute flaccid myelitis (AFM).

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

1. Yip C C, Lau S K, Woo P C, Yuen K Y. Human enterovirus 71 epidemics: what's next? Emerging health threats journal. 2013; 6:19780.
2. Ruuskanen O, Lahti E, Jennings L C, Murdoch D R. Viral pneumonia. Lancet. 2011; 377 (9773): 1264-75.
3. Wong S S, Yip C C, Lau S K, Yuen K Y. Human enterovirus 71 and hand, foot and mouth disease. Epidemiol Infect. 2010; 138 (8): 1071-89.
4. Peters C E, Carette J E. Return of the Neurotropic Enteroviruses: Co-Opting Cellular Pathways for Infection. Viruses. 2021; 13 (2).
5. Chen B S, Lee H C, Lee K M, Gong Y N, Shih S R. Enterovirus and Encephalitis. Front Microbiol. 2020; 11:261.
6. Nishimura Y, Sato K, Koyanagi Y, Wakita T, Muramatsu M, Shimizu H, et al. Enterovirus A71 does not meet the uncoating receptor SCARB2 at the cell surface. Plos Pathog. 2024; 20 (2): e1012022.
7. Wang Y F, Yu C K. Animal models of enterovirus 71 infection: applications and limitations. J Biomed Sci. 2014; 21:31.
8. Yeung M L, Jia L, Yip C C Y, Chan J F W, Teng J L L, Chan K H, et al. Human tryptophanyl-tRNA synthetase is an IFN-gamma-inducible entry factor for Enterovirus. J Clin Invest. 2018.
9. Rosenfeld A B, Warren A L, Racaniello V R. Neurotropism of Enterovirus D68 Isolates Is Independent of Sialic Acid and Is Not a Recently Acquired Phenotype. mBio. 2019; 10 (5).
10. Hooi Y T, Balasubramaniam V. In vitro and in vivo models for the study of EV-D68 infection. Pathology. 2023.
11. Liou A T, Wu S Y, Liao C C, Chang Y S, Change C S, Shih C. A new animal model containing human SCARB2 and lacking stat-1 is highly susceptible to EV71. Scientific Reports. 2016; 6:31151.

What is claimed:

1. A transgenic mouse comprising a hWARS vector that expresses at least a portion of a hWARS gene and functional/structural variants thereof, in one or more tissues and exhibits an increased susceptibility to enterovirus infection as compared to a mouse without the hWARS gene.

2. The mouse of claim 1 wherein the hWARS gene is a wild-type gene.

3. The mouse of claim 1 wherein the hWARS vector is targeted to a mouse ROSA26 gene.

4. The mouse of claim 3 wherein the hWARS vector comprising a CMV promoter-Kozak-human WARS CDS-rBG pA cassette.

5. The mouse of claim 3 wherein the hWARS vector comprises a nucleic acid sequence of SEQ ID NO:1.

6. The transgenic mouse of claim 1 which is a mouse model for enterovirus infection.

7. The transgenic mouse of claim 1 wherein the enterovirus infection is EV-D68, EV-A71 or a combination thereof.

8. The transgenic mouse of claim 1 wherein the enterovirus infection is related to a disease or disorder selected from the group consisting of severe neurological disease, aseptic meningitis, acute flaccid paralysis, fatal neurogenic pulmonary edema, hand, foot, and mouth disease (HFMD), brainstem encephalitis, meningitis, polio-like paralysis, severe respiratory disease, and acute flaccid myelitis (AFM).

9. The transgenic mouse of claim 1 wherein the hWARS gene and functional/structural variants thereof is expressed at least in one tissue comprising brain, muscle, and lung.

10. The transgenic mouse of claim 1 comprising a hWARS knock-in mutation.

11. The transgenic mouse of claim 1, wherein the mouse is treated with a compound which lowers the level of susceptibility to enterovirus infection.

12. The transgenic mouse of claim 1, wherein the enterovirus infection is treated by administering small molecules, antibiotics, antiviral agents, or antibodies.

13. A vector comprising a CMV promoter-Kozak-human WARS CDS-rBG pA cassette.

14. A method of screening for compounds that treat enterovirus infection comprising: (i) administering the compound to a transgenic mouse that expresses a portion of a hWARS gene and functional/structural variants thereof, in one or more tissues with an increased susceptibility of enterovirus infection as compared to that in a wild-type mouse, wherein the mouse that comprises a hWARS knock-in vector; and (ii) determining the effect of the compound on treating enterovirus infection in the transgenic mouse, relative to a control mouse not treated with the compound or a control mouse without the hWARS knock-in vector treated with the compound; and (iii) identifying the compound.

15. A method of treating a disease or condition in a subject in need thereof, said method comprises administering an effective amount of a compound identified in claim 14, said disease or disorder is related to an enterovirus infection, the disease or disorder is selected from the group consisting of severe neurological disease, aseptic meningitis, acute flaccid paralysis, fatal neurogenic pulmonary edema, hand foot and mouth disease (HFMD), brainstem encephalitis, meningitis, polio-like paralysis, severe respiratory disease, and acute flaccid myelitis (AFM).
